(19) United States
(12) Patent Application Publication
(10) Pub. No.: US 2023/0355279 A1
(43) Pub. Date: Nov. 9, 2023

(54) Bone Screw for Attaching Rod to Bone

(71) Applicant: CoreLink, LLC, St. Louis, MO (US)

(72) Inventor: Mike Mueller, Union, MO (US)

(21) Appl. No.: 18/312,303

(22) Filed: May 4, 2023

Related U.S. Application Data
(60) Provisional application No. 63/364,117, filed on May 4, 2022.

(51) Int. Cl. A61B 17/70

(52) U.S. Cl. CPC A61B 17/7032

(57) ABSTRACT

A modular pedicle screw includes a head assembly configured to “pop” or “snap” on a fastener of the screw. The head assembly includes a collet. The collet includes a friction ring for frictionally engaging a head of the fastener. The collet includes ring portions defining a split ring for receiving the head of the fastener. The ring portions are movable axially and radially relative to a rod coupler of the screw to enable attachment and detachment of the head assembly on the head of the fastener. The collet is movable axially relative to a saddle of the screw. The saddle and collet are coupled to one another by a tongue of the collet being received in a groove of the saddle.

### CROSS-REFERENCE TO RELATED APPLICATIONS

The present application claims the benefit of U.S. Provisional Application No. 63/364,117, filed May 4, 2023, the entirety of which is hereby incorporated by reference.

### FIELD OF THE DISCLOSURE

The present disclosure is directed to a bone screw, and in particular, a bone screw for use in attaching a rod to bone, such as a vertebra.

### BACKGROUND OF THE DISCLOSURE

Bone screws may be used to attach or anchor a rod to a bone. For example, spinal bone screws may be used to anchor implants to vertebrae along the spinal column for the purpose of stabilizing and/or adjusting spinal alignment. Spinal fusion surgery may require placement of bone screws through small incisions with limited visibility for connecting vertebrae to rods. Due to the difficulty in placing bone screw in such procedures, polyaxial screw are often used. Polyaxial screw assemblies allow for rotational and pivotable movement of a receiver about the head of a fastener until a desired position of the receiver is achieved. A rod or other connecting member may then be inserted into a channel of the receiver and secured in place by a locking member engaged with the receiver.

### SUMMARY

In one aspect, a spinal pedicle screw having proximal and distal ends and an axis extending therebetween generally comprises: a fastener comprising a shaft and a fastener head disposed on the shaft, the fastener being configured to be anchored within a subject's bone; and a head assembly coupled to the fastener head. The head assembly includes a rod coupler configured to secure a rod to the head assembly, and a collet received at least partially within the rod coupler. The fastener head is received in the collet. The collet has an interior surface engaging the fastener head. The collet includes a friction rib on the interior surface and protruding inward therefrom. The friction rib is configured to impart a frictional force on the fastener head to inhibit free movement of the head assembly on the fastener head.

In another aspect, a spinal pedicle screw having opposite proximal and distal ends and an axis extending therebetween generally comprises: a fastener comprising a shaft and a fastener head disposed on the shaft, the fastener being configured to be anchored within a subject's bone; and a head assembly. The head assembly includes a rod coupler configured to secure a rod to the head assembly. A collet of the head assembly is received at least partially within the rod coupler. The collet has an interior surface engaging the fastener head and at least one tongue extending radially inward from the interior surface. A saddle of the head assembly is received at least partially within the rod coupler. The saddle has a proximal surface for seating the rod thereon. The saddle defines at least one exterior groove. The at least one tongue of the collet is received the at least one groove of the saddle to couple the collet to the saddle. The collet and the saddle are movable axially relative to one another along the axis of the spinal pedicle screw.

In yet another aspect, a head assembly for a spinal pedicle screw having opposite proximal and distal ends and an axis extending therebetween generally comprises: a rod coupler configured to secure a rod to the head assembly; a collet received at least partially within the rod coupler, wherein the collet has an interior surface configured to engage the fastener head; and a saddle received at least partially within the rod coupler, the saddle having a proximal surface for seating the rod thereon. Together the collet and the saddle define a cavity sized and shaped to receive a fastener head of the fastener. The collet and the saddle are movable axially relative to one another along the axis of the spinal pedicle screw. The collet includes ring portions that are free from direct securement to one another and are in generally opposing relationship to effectively form a split ring. The ring portions are simultaneously movable axially and radially relative to the rod coupler to enable the fastener head of the fastener to be inserted into the collet as the head assembly is pushed down on the fastener head.

### BRIEF DESCRIPTION OF THE DRAWINGS

FIG. 1 is a perspective of an embodiment of a bone screw for attaching a rod to a bone.

FIG. 2 is an exploded view of the bone screw.

FIG. 3 is an enlarged, fragmentary cross section of the bone screw taken through a first longitudinal plane.

FIG. 4 is a similar to FIG. 3 except taken through a second longitudinal plane at a 90 degree angle relative to the first longitudinal plane.

FIG. 5 is an enlarged perspective of a coupled fastener collet and a saddle of the bone screw.

FIG. 6 is a front elevational view of the coupled fastener collet and a saddle, the rear elevational view being identical.

FIG. 7 is a right elevational view of the coupled fastener collet and a saddle, the left elevational view being identical.

FIG. 8 is a cross section taken through the line 8-8 in FIG. 6.

FIG. 9 is an enlarged, fragmentary cross section of a fastener of the bone screw being inserted into a head assembly of the bone screw through a distal opening of a rod coupling of the head assembly prior to the head assembly being coupled to the fastener.

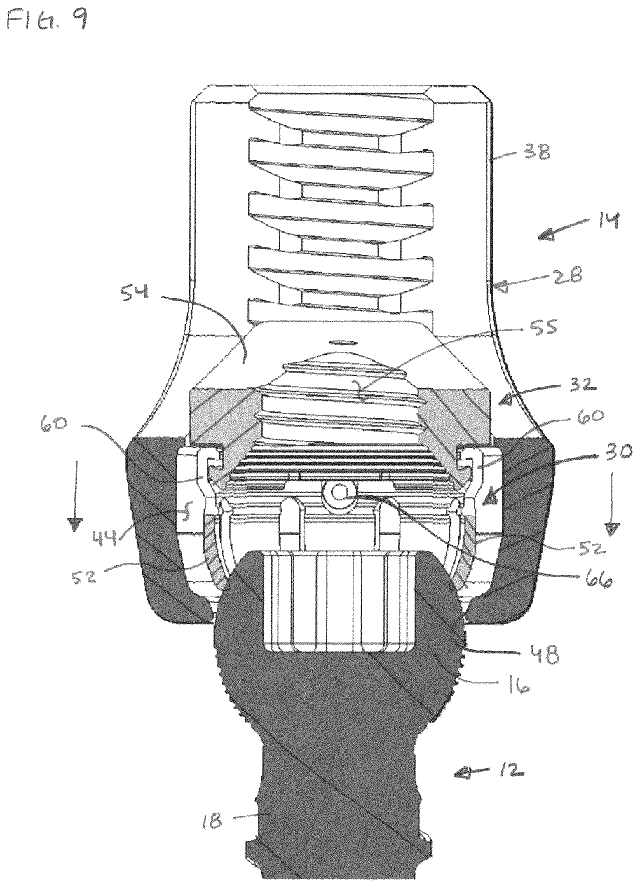

FIG. 10 is a perspective of another embodiment of a bone screw.

FIG. 11 is an exploded view of the bone screw.

FIG. 12 is a perspective of one ring portion of a collet of the bone screw.

FIG. 13 is an exploded view showing ring portions of the collet removed from a saddle of the bone screw.

FIG. 14 is similar to FIG. 13, with the components shown in cross section.

FIG. 15 is a perspective of the collet received on the saddle and showing pins received in openings or slots of the collet.

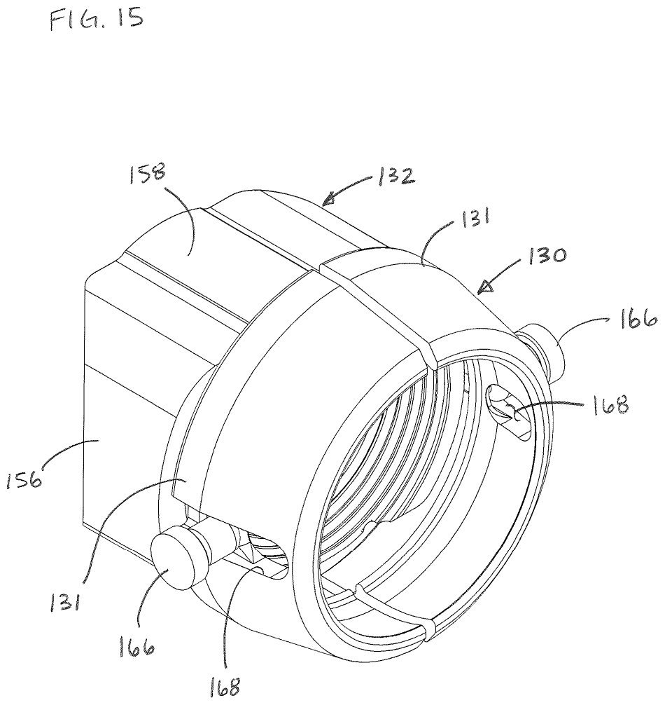

FIG. 16 is a side elevational view of the collet received on the saddle.

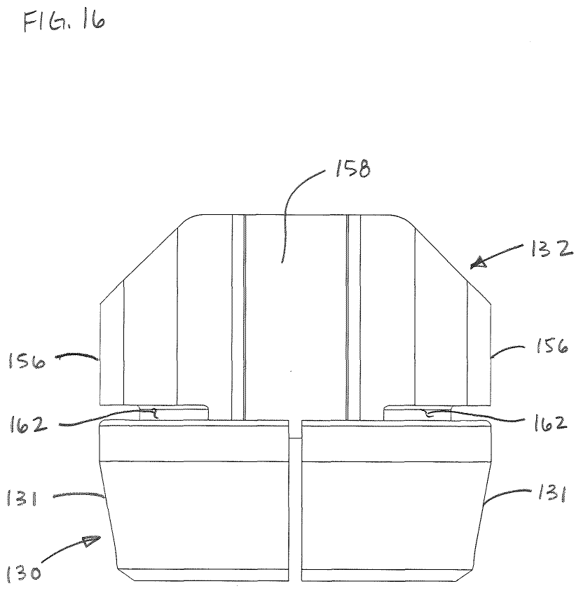

FIG. 17 is a front elevational view of the collet received on the saddle.

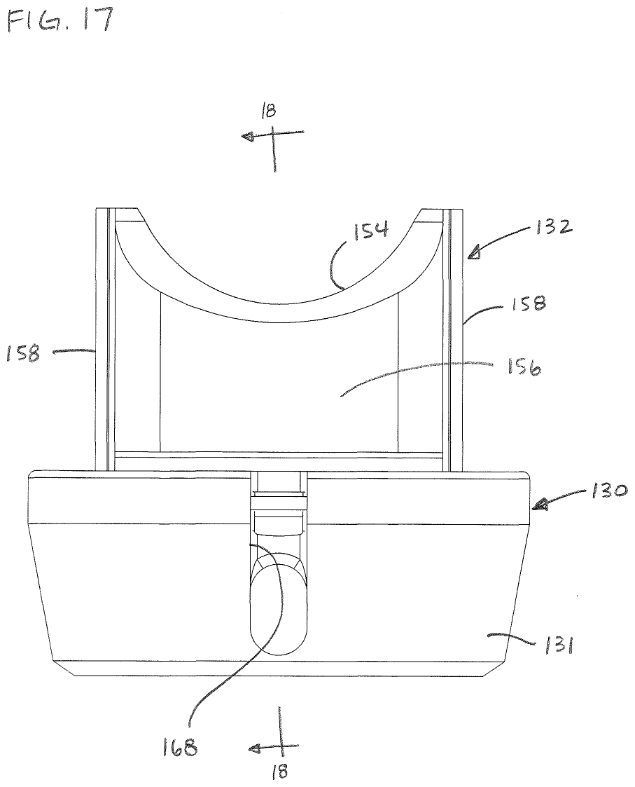

FIG. 18 is a cross section of FIG. 17 taken through the plane 18-18.

FIG. 19 is a perspective showing one of the ring portions of the collet being inserted into a rod coupler of the screw before inserting the saddle.

FIG. 20 is a perspective showing the saddle being inserting into the rod coupler after inserting the collet.

FIG. 21 is an enlarged partial front elevational view showing a fastener head of the screw being inserted into a head assembly of the screw.

FIG. 22 is a cross section of FIG. 21 taken through the plane 22-22.

FIG. 23 is an enlarged partial front elevational view showing the fastener head of the screw fully received in the head assembly in a proximal-most position.

FIG. 24 is a cross section of FIG. 23 taken through the plane 24-24.

FIG. 25 enlarged partial front elevational view showing the fastener head of the screw being secured to the head assembly.

FIG. 26 is a cross section of FIG. 25 taken through the plane 26-26.

Corresponding reference characters indicate corresponding parts throughout the drawings.

### DETAILED DESCRIPTION OF THE DISCLOSURE

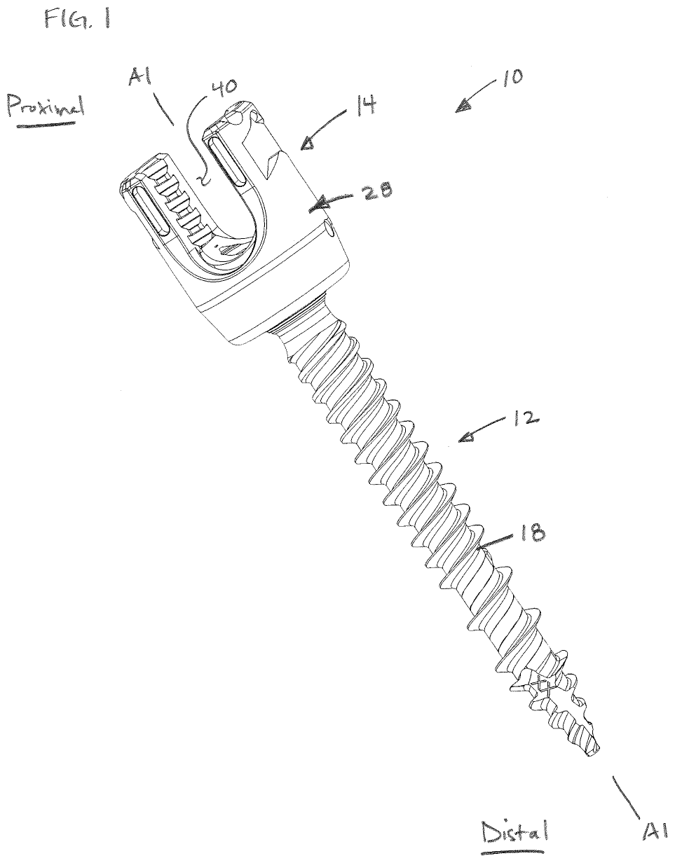

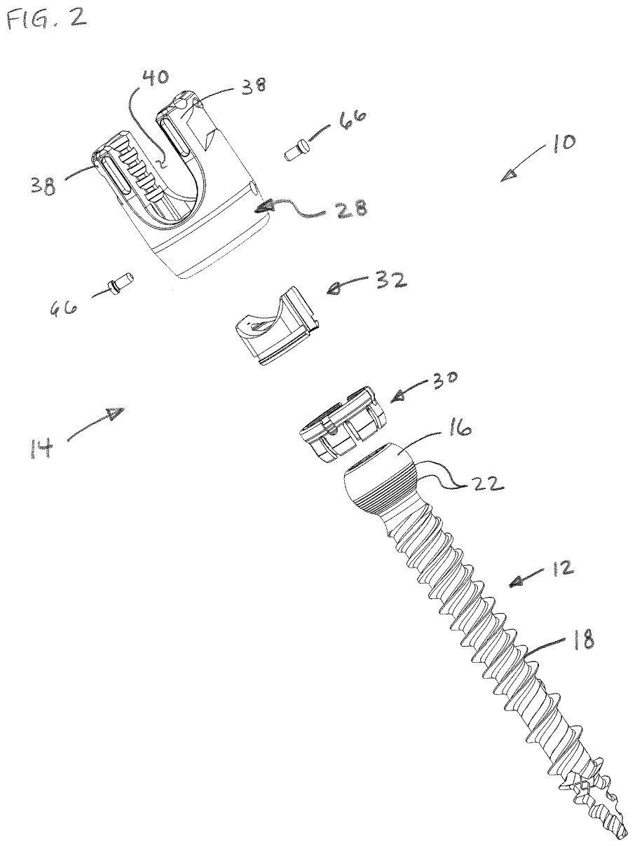

Referring to FIGS. 1 and 2 of the drawings, an embodiment of a bone screw constructed according to the teachings of the present disclosure is generally indicated at reference numeral 10. The illustrated bone screw 10 is suitable for use as a spinal pedicle bone screw, and in particular, a polyaxial pedicle bone screw for use in attaching or anchoring a spinal rod (e.g., a titanium rod) to adjacent vertebrae. This type of system, including the bone screw 10 and the rod, may be used in spinal fusion procedures, which are generally known in the art. The bone screw 10 generally includes a fastener, generally indicated at 12, (e.g., a threaded fastener, such as a screw) to anchor the bone screw to a subject's bone (e.g., vertebra), and a head assembly (e.g., a tulip head assembly), generally indicated at 14, configured to be removably coupled to the fastener and to couple a spinal rod to the bone screw. The illustrated bone screw 10 is a “bottom-loading” type pedicle screw because the bone screw is assembled by inserting the fastener through a bottom of the head assembly 14, whereby the head assembly snaps on or otherwise couples to the fastener. The bone screw 10 has proximal and distal end portions and an axis A1 extending therebetween. As used herein, the terms “proximal” and “distal” are used to denote relative locations of components and structures of the bone screw, and the term “proximal” relates to the upper end of the bone screw as orientated in FIG. 1 and the term “distal” relates to the lower end of the bone screw as orientated in FIG. 1.

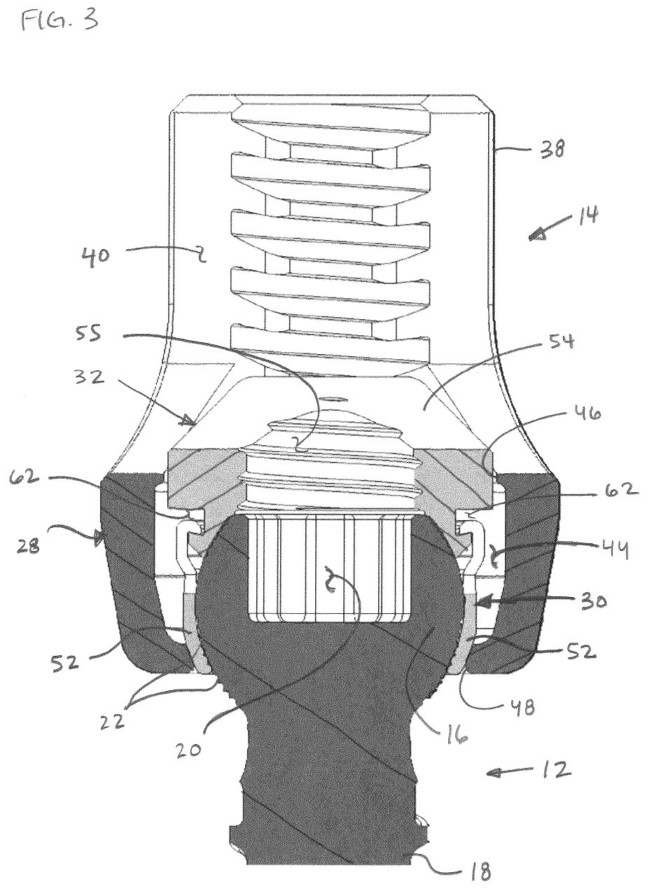

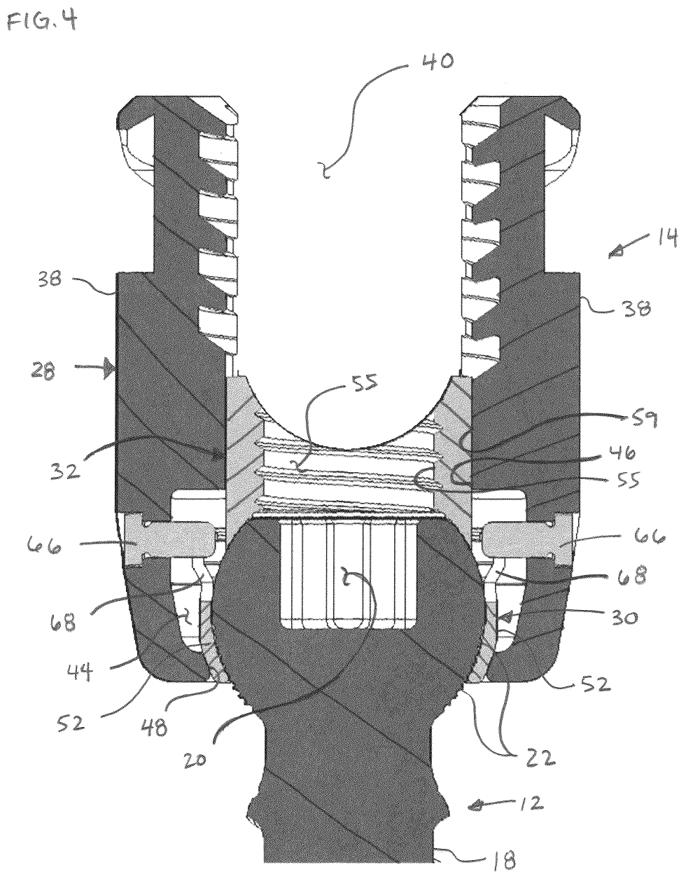

The illustrated fastener 12 is an exemplary fastener for use with the head assembly 14. The fastener 12 includes a threaded shaft 18 extending distally outward from a head 16 of the fastener. The threads on the threaded shaft 18 are suitable for threading into a bone, such as vertebra, to anchor the fastener 12 to the bone. In other embodiments, the shaft 18 may not be threaded but may be otherwise configured anchor the fastener 12 to the bone. Referring to FIGS. 2-4, the fastener head 16 is spherical or ball-shaped with a truncated proximal end. A socket 20 (FIGS. 3 and 4) for receiving a tool head (e.g., a driver; not shown) extends distally from the truncated proximal end. A distal portion of the fastener head 16 includes ribs 22 extending circumferentially around the fastener head and being spaced apart from one another along the axis A1 of the bone screw 10. A proximal portion of the fastener head 16 is free from ribs and may have a generally smooth exterior surface. In the illustrated embodiment, the distal longitudinal portion of the head 16 encompasses about one half of the axial length of the fastener head 16, and the proximal longitudinal portion of the head encompasses the remainder (about one half) of the axial length of the fastener head. As explained below, the fastener head 16 is configured to be received in the head assembly 14 to couple the head assembly to the fastener 12. The fastener 12 may be formed from metal, such as titanium, or other material. As will be explained in more detail below, the illustrated head assembly 14 may be modular and configured to be coupled to any one of a plurality of fasteners, where each of the fasteners has the same fastener head 16 but may have different shaft lengths, shaft diameters, thread diameters, thread pitches, etc.

Referring to FIGS. 2-4, the head assembly 14 includes a rod coupler 28, and a fastener collet 30 and rod saddle 32 received in the rod coupler, each indicated generally. The illustrated rod coupler 28 is a tulip coupler including proximally extending arms 38 defining a slot 40 (e.g., U-shaped slot) therebetween. The slot 40 is sized and shaped to receive a rod (e.g., spinal rod) therein. Interior surfaces of the arms 38 are threaded to threadably secure a cap (not shown) within the rod coupler 28. The cap presses down on the rod received between the arms 38 to secure the rod to the head assembly 14 and lock the head assembly 14 on the fastener head 16, as explained below. A distal portion of the rod coupler 28 defines an expansion cavity 44, a proximal opening 46 at a proximal end of the expansion cavity, and a distal opening 48 at a distal end of the expansion cavity and extending through the distal end of the rod coupler. As can be seen in FIGS. 3 and 4, a major diameter of the expansion diameter 44 is greater than a diameter of the distal opening 48 of the rod coupler 28. The distal opening 48 is sized and shaped to enable the fastener head 16 to be inserted therethrough into the expansion cavity 44. The expansion cavity 44 is sized and shaped to receive at least the fastener collet 30 therein to enable expansion of the collet and coupling of the collet to the fastener head 16, as explained below.

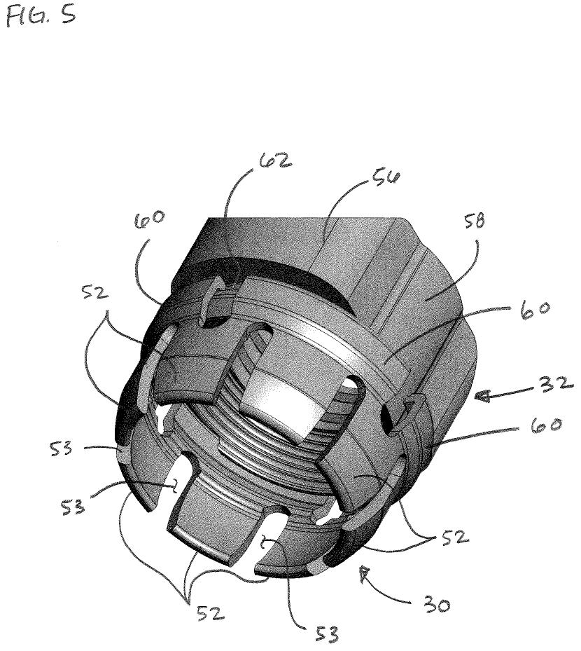

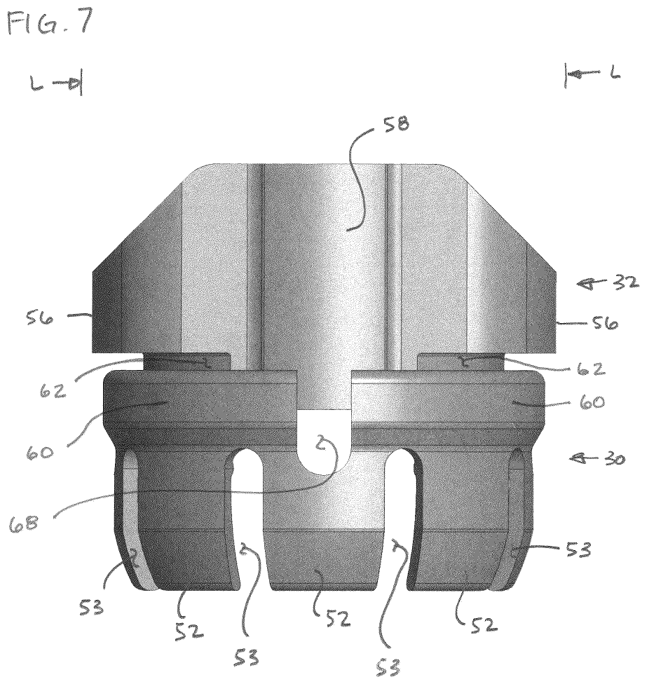

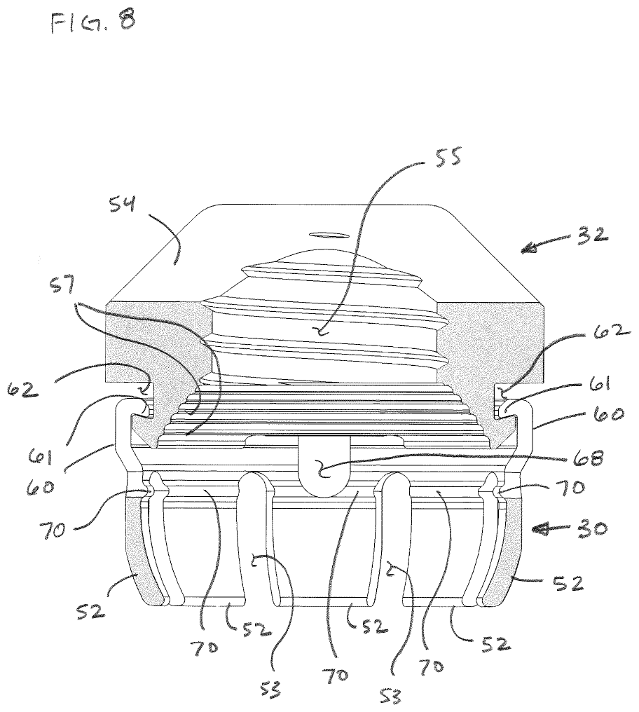

Referring to FIGS. 5-7, the fastener collet 30 includes distal portion having resiliently deflectable clamp arms 52 circumferentially spaced apart from one another around an outer diameter of the collet, and open slots 53 disposed between adjacent ones of the clamp arms 52. The clamp arms 52 are configured to snap on the fastener head 16, as explained in more detail below, such that interior surfaces of the clamp arms engage the fastener head. A distal portion of the rod saddle 32 defines an interior surface that engages the fastener head 16 when the fastener head is snapped on the collet 30. Together, the interior surfaces of the clamp arms 52 and the distal portion of the rod saddle 32 effectively define a generally round or spherical head cavity for receiving the fastener head 16. In the illustrated embodiment, as seen best in FIG. 8, the interior surfaces of the clamp arms 52 are generally smooth (e.g., non-ribbed) and engage the ribs 22 on the distal portion of the fastener head; and the interior surface of the distal portion of the rod saddle 32 includes longitudinally spaced ribs 57 extending circumferentially around the interior surface and engage the smooth proximal portion of the fastener head.

The rod saddle 32 includes a proximal portion defining a seat 54 on which the rod engages or seats when received in the slot 40 of the rod coupler 28. As shown in FIGS. 3, 4 and 8, the saddle 32 defines a tool-receiving opening 55 extending axially through the seat 54. The illustrated opening 55 is threaded so that a tool can be threadably coupled to the saddle 32, as explained in more detail below. In the illustrated embodiment, the opening 55 extends through the interior surface that engages the fastener head 16 so that the driver tool can be inserted through the saddle and coupled to the socket of the fastener. The saddle 32 also includes opposite ends 56 defining a length of the saddle, and opposite sides 58 defining a width of the saddle that is less than the length. As shown in FIG. 4, the rod coupler 28 defines a saddle-receiving recess 59 having a width slightly larger than the width of the saddle 32 to inhibit rotation of the saddle relative to the rod coupler. The saddle 32 is capable of moving axially (in the proximal-distal direction) within the rod coupler 28. This axial is in the proximal direction is restricted by an internal stop defined by the rod coupler 28 engaging the saddle 32, and is restricted in the distal direction by the collet 30, for example.

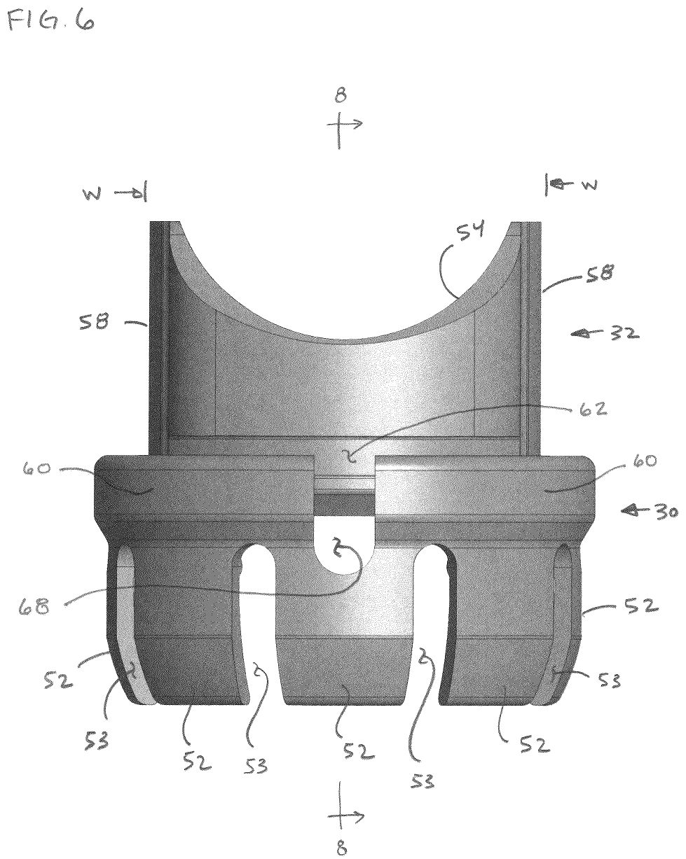

Referring to FIGS. 6-8, a proximal portion of the collet 30 includes resiliently deflectable snap-fit arms 60 that are snap-fitted on a distal portion of the saddle 32. In particular, the snap-fit arms 60 are cantilevered and include a protrusion 61 (broadly, a tongue, e.g., hood, bead, or lug, etc.) configured to be captured in an exterior annular or arcuate groove 62 of the saddle 32. In the illustrated embodiment, the groove 62 is discontinuous and defined by the ends 56 of the saddle but not by the sides 58 of the saddle. As seen in FIG. 8, the distal portion of the saddle 32 is at least partially received in the collet 30, in particular, within the proximal portion of the collet. The protrusions 61 are longitudinally slideable within the groove 62 of the saddle such that the collet 30 and the saddle 32 are longitudinally movable relative to one another. Because the protrusions 61 are captured and retained in the groove 62, upper and lower ends of the groove 62 restrict the longitudinal movement collet 30 and the saddle 32 relative to one another. As seen in FIG. 4, pins 66 (e.g., two diametrically opposite pins) extend though the rod coupler 28 and into radial openings 68 defined by the collet 30 to inhibit the collet from rotating relative to the rod coupler and the saddle 30.

Referring to FIG. 8, the collet 30 includes a friction rib 70 on the interior surface thereof. In particular, the illustrated friction rib 70 protrudes inward from the interior surfaces of the clamping arms 52. The rib 70 is discontinuous and adjacent proximal ends of the clamping arms. The rib 70 may generally circumscribe the interior surfaces of the clamping arms to define a discontinuous ring. In use, the rib 70 frictionally engages the fastener head 16 (e.g., the distal portion of the fastener head) to provide frictional force against polyaxial movement of the collet on the fastener head when the fastener head is received in the spherical cavity defined by the collet and the saddle before the rod is fastened to the screw. The rib 70 enables polyaxial movement while providing the frictional force to inhibit free polyaxial movement of the head assembly relative to the fastener (such as due to gravitational forces) before the rod is secured to the bone screw.

To secure the head assembly 14 to the fastener, the head of the fastener is inserted into distal opening of the rod coupler. As the fastener head is moved axially into the distal opening, the collet 30 and the saddle 32 are pushed proximally by the fastener head and the collet moves proximally relative to the saddle, as explained above. As the fastener head 16 continues to advance axially, the clamping arms 52 are moved into the expansion cavity 44 of the rod coupler 28 so that the fastener head 16 resiliently deflects the clamping arms radially outward and the fastener head enters (snaps into) the collet 30 and the head cavity in general inside the expansion cavity. With the fastener head 16 in the head cavity, the saddle 32 and the collet 30 are moved distally (such as by using the tool secured to the saddle) so that the distal ends of the clamping arms 52 enter the distal opening 48 of the rod coupler 28 and are pinched against the fastener head 16. In this position, the head assembly 14 is capable of polyaxial movement relative to the fastener head 16 against the frictional forces of the friction rib 70 acting on the fastener head 16. The head assembly 14 is also capable of being removed from the fastener 12 using the tool by moving the saddle 32 and the collet 30 proximally relative to the rod coupler 28 such that the clamping arms 52 enter the expansion cavity. With the clamping arms 52 in the expansion cavity 44, the head assembly 14 can be moved proximally relative to the fastener head, causing the clamping arms 52 to radially deflect outward so that the fastener head is removed from the collet and the head assembly unsnaps or releases from the fastener head.

The head assembly 14 is fixed to the fastener 12 and inhibited from polyaxial movement when the rod is secured to the screw 10, such as by threading the cap in the rod coupler 28. Securing the rod to the screw 10 pushes the saddle 32 against the fastener head 16 and further compresses the clamping arms 52 around the fastener head to inhibit movement of the head assembly 14 relative to the fastener head. The head assembly 14 may be secured to the fastener before anchoring the fastener in bone or after anchoring the fastener in bone.

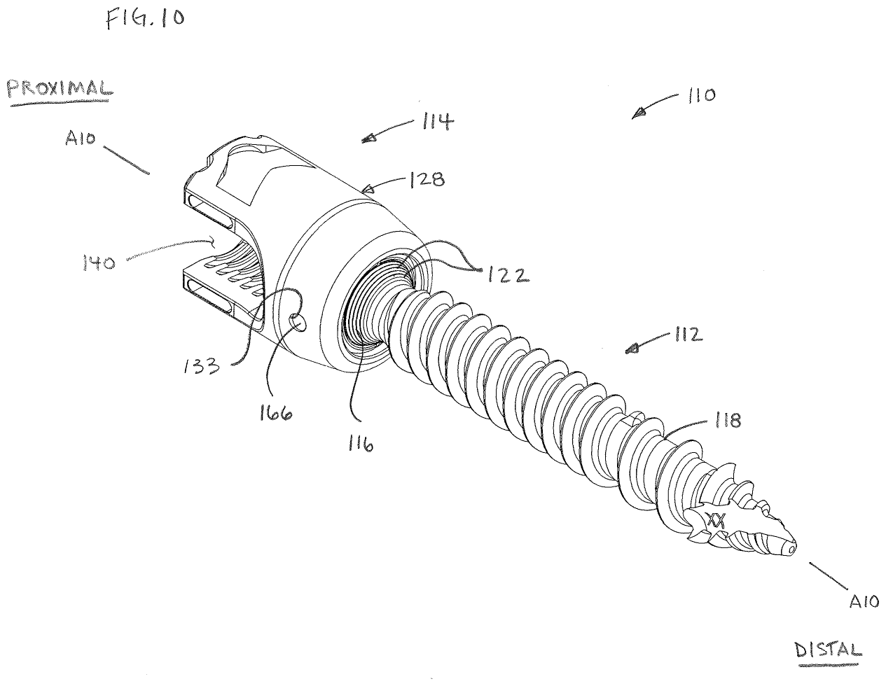

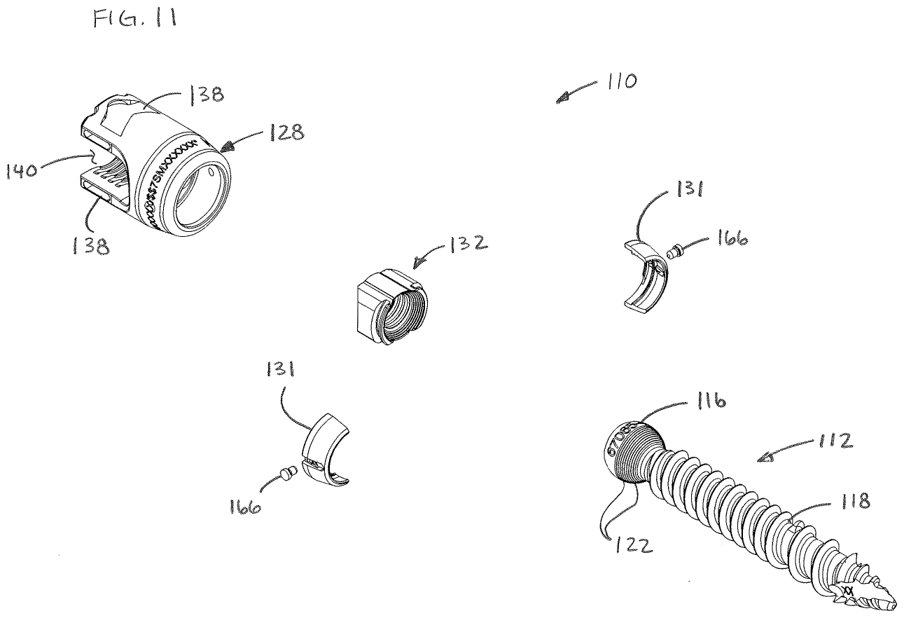

Referring to FIGS. 10 and 11 of the drawings, another embodiment of a bone screw constructed according to the teachings of the present disclosure is generally indicated at reference numeral 110. The illustrated bone screw 110 is suitable for use as a spinal pedicle bone screw, and in particular, a polyaxial pedicle bone screw for use in attaching or anchoring a spinal rod (e.g., a titanium rod; not shown) to adjacent vertebrae. This type of system, including the bone screw 110 and the rod, may be used in spinal fusion procedures, which are generally known in the art. The bone screw 110 generally includes a fastener, generally indicated at 112, (e.g., a threaded fastener, such as a screw) to anchor the bone screw to a subject's bone (e.g., vertebra), and a head assembly (e.g., a tulip head assembly), generally indicated at 114, configured to be removably coupled to the fastener and to couple a spinal rod to the bone screw. The illustrated bone screw 110 is a “bottom-loading” type pedicle screw because the bone screw is assembled by inserting the fastener through a bottom of the head assembly 114, whereby the head assembly snaps on or otherwise couples to the fastener. The bone screw 110 has proximal and distal end portions and an axis A10 extending therebetween. As used herein, the terms “proximal” and “distal” are used to denote relative locations of components and structures of the bone screw, and the term “proximal” relates to the upper end of the bone screw as orientated in FIG. 10 and the term “distal” relates to the lower end of the bone screw as orientated in FIG. 10.

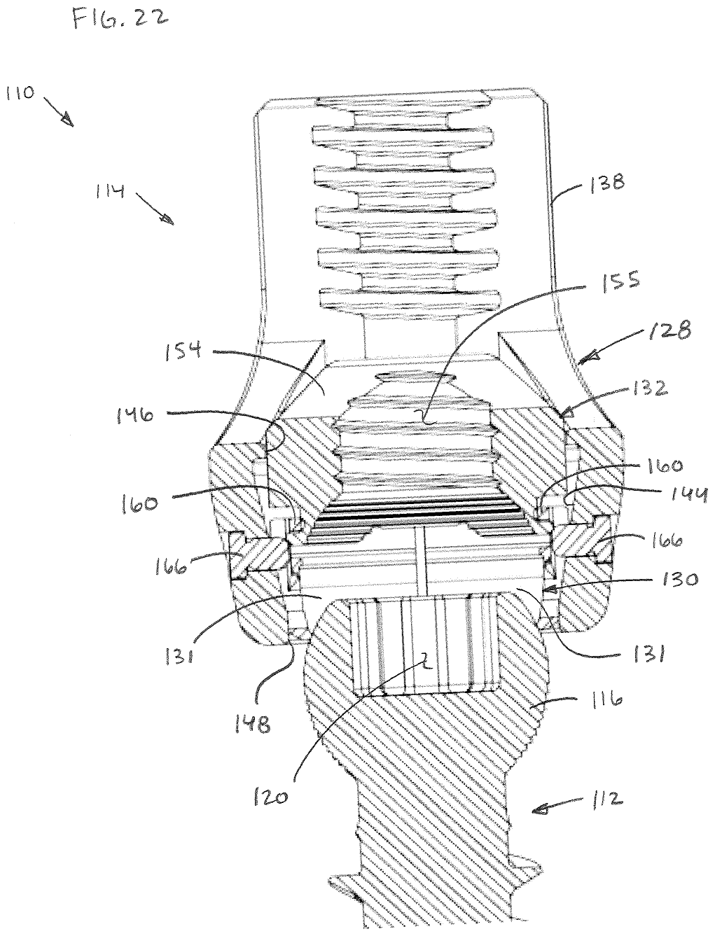

The illustrated fastener 112 is an exemplary fastener for use with the head assembly 114. The fastener 112 includes a threaded shaft 118 extending distally outward from a head 116 of the fastener. The threads on the threaded shaft 118 are suitable for threading into a bone, such as vertebra, to anchor the fastener 112 to the bone. In other embodiments, the shaft 118 may not be threaded but may be otherwise configured anchor the fastener 112 to the bone. Referring to FIG. 11, the fastener head 116 is spherical or ball-shaped with a truncated proximal end. A socket 120 (FIGS. 22, 24 and 26) for receiving a tool head (e.g., a driver; not shown) extends distally from the truncated proximal end. A distal portion of the fastener head 116 includes ribs 122 extending circumferentially around the fastener head and being spaced apart from one another along the axis A10 of the bone screw 110. A proximal portion of the fastener head 16 is free from ribs and may have a generally smooth exterior surface. In the illustrated embodiment, the distal longitudinal portion of the head 116 encompasses about one half of the axial length of the fastener head 116, and the proximal longitudinal portion of the head encompasses the remainder (about one half) of the axial length of the fastener head. As explained below, the fastener head 116 is configured to be received in the head assembly 114 to couple the head assembly to the fastener 112. The fastener 112 may be formed from metal, such as titanium, or other material. As will be explained in more detail below, the illustrated head assembly 114 may be modular and configured to be coupled to any one of a plurality of fasteners, where each of the fasteners has the same fastener head 116 but may have different shaft lengths, shaft diameters, thread diameters, thread pitches, etc.

Referring sill to FIGS. 10 and 11, the head assembly 114 includes a rod coupler 128, and a fastener collet 130 and rod saddle 132 received in the rod coupler, each indicated generally. The illustrated rod coupler 128 is a tulip coupler including proximally extending arms 138 defining a slot 140 (e.g., U-shaped slot) therebetween. The slot 140 is sized and shaped to receive a rod (e.g., spinal rod) therein. Interior surfaces of the arms 138 are threaded to threadably secure a cap (not shown) within the rod coupler 128. The cap presses down on the rod received between the arms 138 to secure the rod to the head assembly 114 and lock the head assembly on the fastener head 116, as explained below. As shown in FIGS. 22, 24 and 26, a distal portion of the rod coupler 128 defines an expansion cavity 144, a proximal opening 146 at a proximal end of the expansion cavity, and a distal opening 148 at a distal end of the expansion cavity and extending through the distal end of the rod coupler. As can be seen, a major diameter of the expansion diameter 144 is greater than a diameter of the distal opening 148 of the rod coupler 128, and the inner diameter of the expansion cavity 14 (i.e., the interior surface defining the expansion cavity) tapers distally. The distal opening 148 is sized and shaped to enable the fastener head 116 to be inserted therethrough into the expansion cavity 144. The expansion cavity 144 is sized and shaped to receive at least the fastener collet 130 therein to enable radial expansion of the collet (as explained below), and coupling of the collet to the fastener head 116, as explained below.

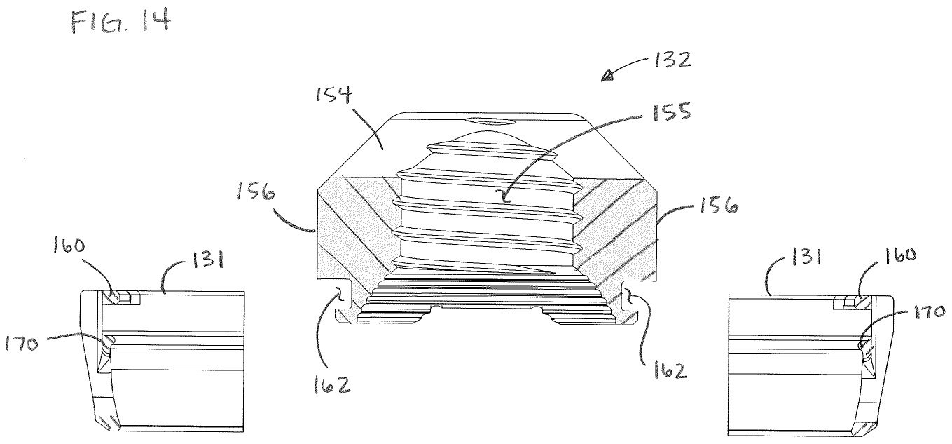

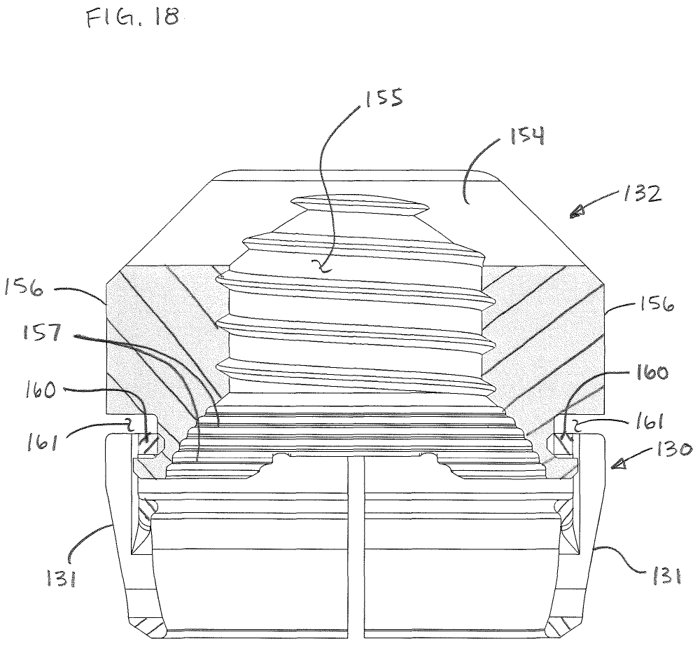

Referring to FIGS. XX, the fastener collet 130 includes two or more ring portions 131 (e.g., two ring portions) defining a circumferential portion of a split ring. The ring portions 131 are free from direct securement to one another and are in generally opposing relationship to effectively form the split ring. The ring portions 131 may be substantially identical to one another in structure and design. As explained in more detail below, the ring portions 131 are movable in the proximal-distal direction and radially inward and outward relative to the saddle 132 (and within the rod coupler 128) to enable a distal portion of the collet 130 to snap on the fastener head 116, such that interior surface of the collet engage the fastener head. The interior surface of the distal portion of the collet 130 may be generally concave or arcuate for acting on the rounded surface of the fastener head 116. The exterior surface of the distal portion of the collet 130 may taper distally for acting on the tapering interior surface of the expansion cavity 144. A distal portion of the rod saddle 132 defines an interior surface that engages the fastener head 116 when the fastener head is snapped on the collet 130. Together, the distal portion of the collet and the distal portion of the rod saddle 132 effectively define a generally round or spherical head cavity for receiving the fastener head 116. In the illustrated embodiment, as seen best in FIGS. 14 and 18, the interior surfaces of the distal portion of the collet 130 are generally smooth (e.g., non-ribbed) and engage the ribs 122 on the distal portion of the fastener head; and the interior surface of the distal portion of the rod saddle 132 includes longitudinally spaced ribs 157 extending circumferentially around the interior surface and engage the smooth proximal portion of the fastener head.

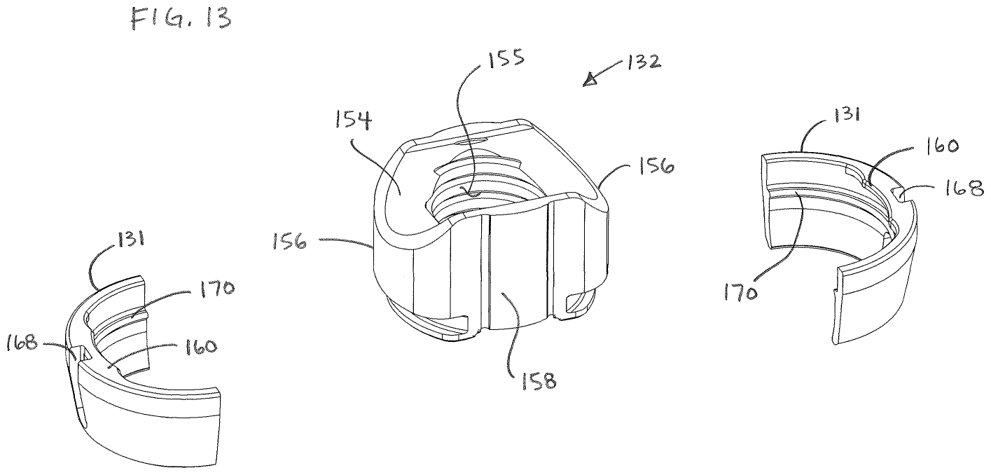

The rod saddle 132 includes a proximal portion defining a seat 154 on which the rod engages or seats when received in the slot 140 of the rod coupler 128. As shown in FIGS. X, the saddle 132 defines a tool-receiving opening 155 extending axially through the seat 154. The illustrated opening 155 is threaded so that a tool can be threadably coupled to the saddle 132, as explained in more detail below. In the illustrated embodiment, the opening 155 extends through the interior surface that engages the fastener head 116 so that the driver tool can be inserted through the saddle and coupled to the socket of the fastener. The saddle 132 also includes opposite ends 156 defining a length of the saddle, and opposite sides 158 defining a width of the saddle that is less than the length. As shown in FIG. 13, the rod coupler 128 defines a saddle-receiving recess 159 having a width slightly larger than the width of the saddle 132 to inhibit rotation of the saddle relative to the rod coupler. The saddle 132 is capable of moving axially (in the proximal-distal direction) within the rod coupler 128. This axial movement in the proximal direction is restricted by an internal stop defined by the rod coupler 128 engaging the saddle 132, and is restricted in the distal direction by the collet 130, for example.

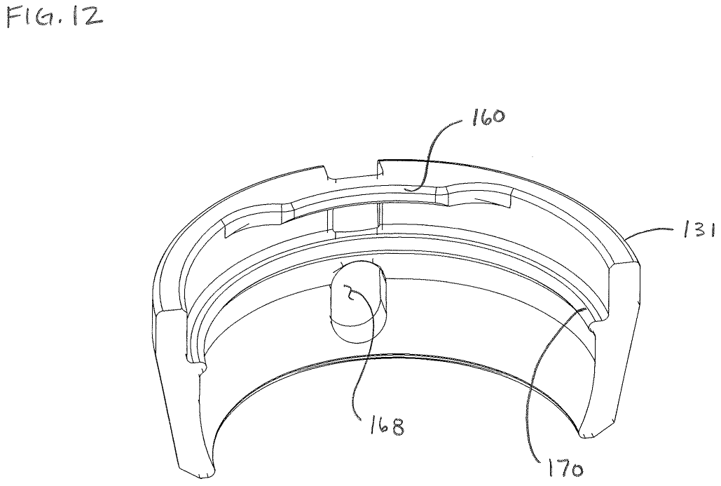

Referring to FIGS. 12-14 and 18, a proximal portion of the collet 130 includes one or more tongues 160 configured to be received and retained in one or more exterior arcuate groove 162 of the saddle 132. In the illustrated embodiment, each of the ring portions 131 includes one or more tongues 160 and the groove 162 includes two grooves defined by the ends 56 of the saddle but not by the sides 58 of the saddle. The tongues 160 are axially (i.e., along the axis of the screw 10) slideable or movable within the groove 162 of the saddle such that the collet 130 and the saddle 132 are axially movable relative to one another along the axis A10. Because the tongues 160 are captured and retained in the groove 162, upper and lower surfaces (i.e., proximal and distal surfaces) defining the groove 162 restrict the axial movement of the collet 130 and saddle 132 relative to one another. As seen in FIGS. 22, 24 and 26, pins 166 (e.g., two diametrically opposite pins) extend through diametrically opposite pin openings 133 defined by the rod coupler 128 and into diametrically opposite radial openings or slots 168 defined by the collet 130 to inhibit or restrict the collet from rotating relative to the rod coupler and the saddle 130. The slots 168 are sized and shaped to enable the axial movement (along axis A10) of the collet 130 relative to the saddle 132.

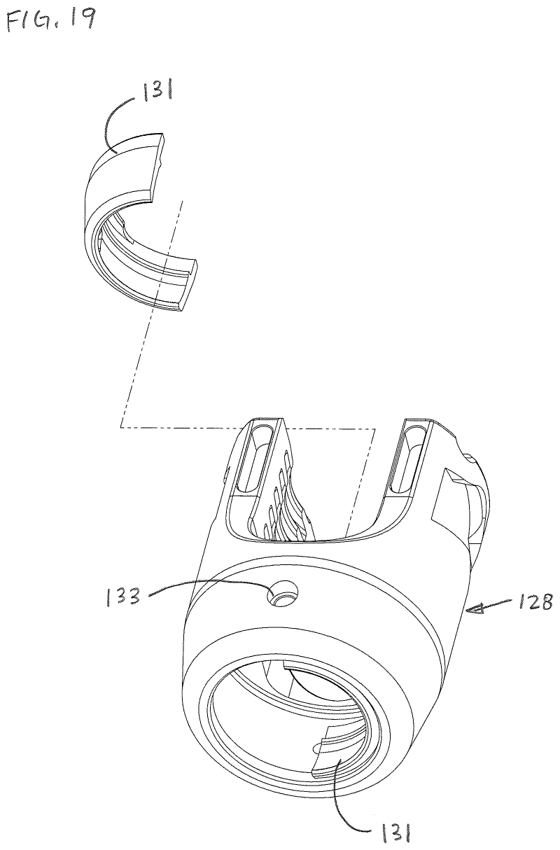

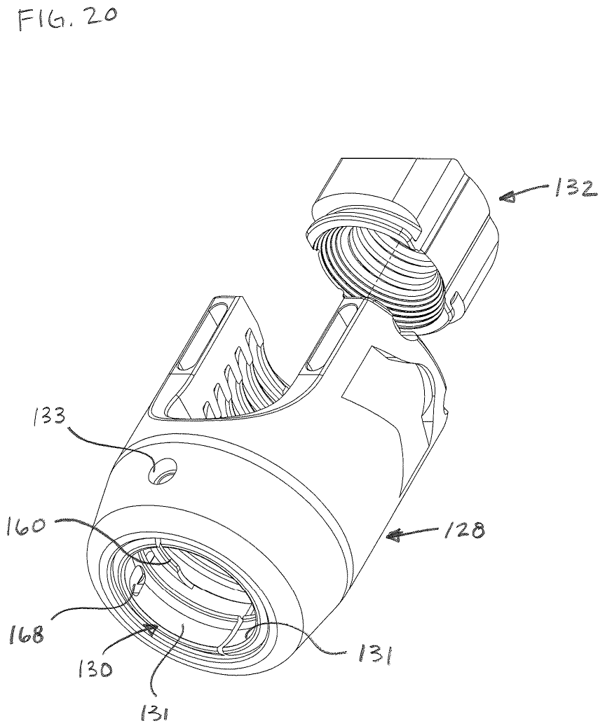

Referring to FIG. 19, to assembly the head assembly 114, the ring portions 131 of the collet 130 are inserted through the proximal opening 146 of the rod coupler 128 in a first angular orientation relative to the rod coupler, in which the slots 168 (and tongues 160) are 90 degrees offset from the pin openings 133. As shown in FIG. 20, the saddle 132 is then inserted into the rod coupler 128 through the proximal opening 146 of the rod coupler 128 and into the proximal portion of the collet 130 through the open proximal end of the collet. After inserting the saddle 132, the collet 130 (i.e., the ring portions 131) is rotated 90 degrees about the axis A10 relative to the saddle 132, whereby the tongues 162 enter the respective grooves 162 and the slots 168 are radially aligned with the pin openings 133. The pins 166 are then inserted into the aligned pin openings 133 and slots 168. The pins 166 are secured to the rod coupler 128. As assembled, the distal portion of the saddle 132 is received in the proximal portion of the collet 130.

Referring to FIG. 8, the collet 130 includes a friction rib 170 on the interior surface thereof. In particular, the illustrated friction rib 170 protrudes inward from the interior surfaces of the ring portions 131. Each ring portion 131 includes a portion of the friction rib 170. The portion of the friction rib 170 may extend along an entirety or substantially and entirety of the arc length of the ring portions 131. As such, the portions of the rib 170 are aligned and may generally circumscribe the interior surface of the collet to define a ring. In use, the rib 170 frictionally engages the fastener head 116 (e.g., the distal portion of the fastener head) to provide frictional force against polyaxial movement of the collet 130 on the fastener head when the fastener head is received in the spherical cavity defined by the collet and the saddle 132 before the rod is fastened to the screw 110. The rib 170 enables polyaxial movement while providing the frictional force to inhibit free polyaxial movement of the head assembly relative to the fastener (such as due to gravitational forces) before the rod is secured to the bone screw 110.

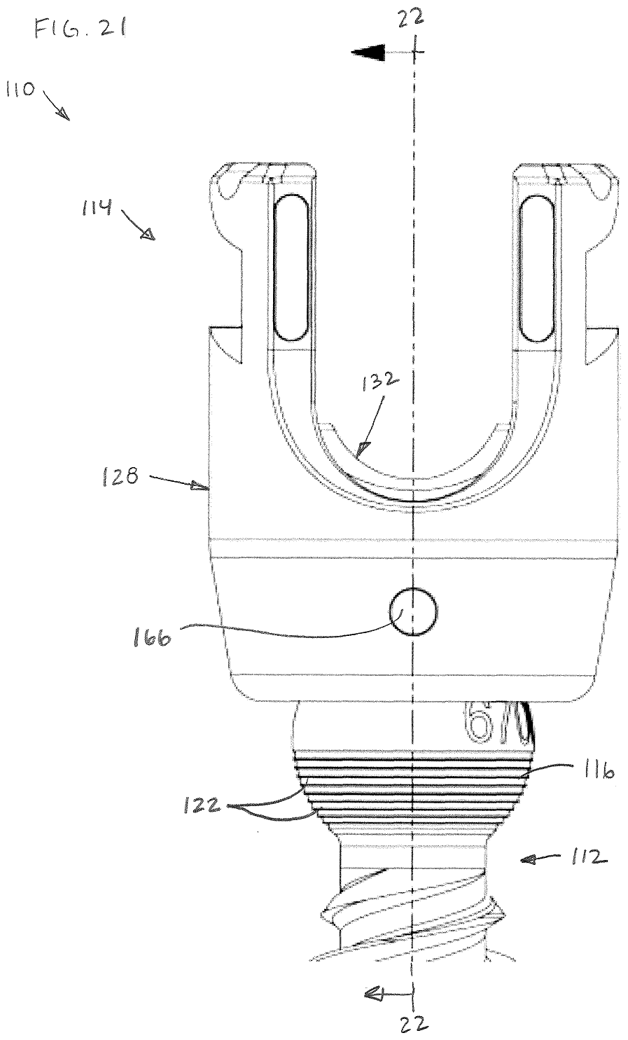

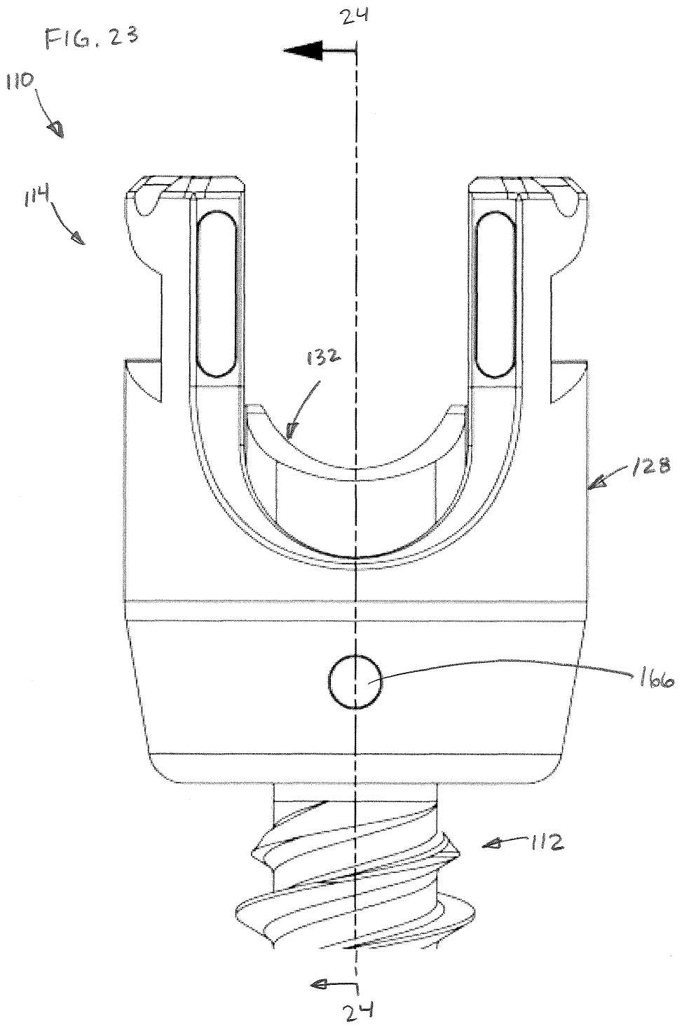

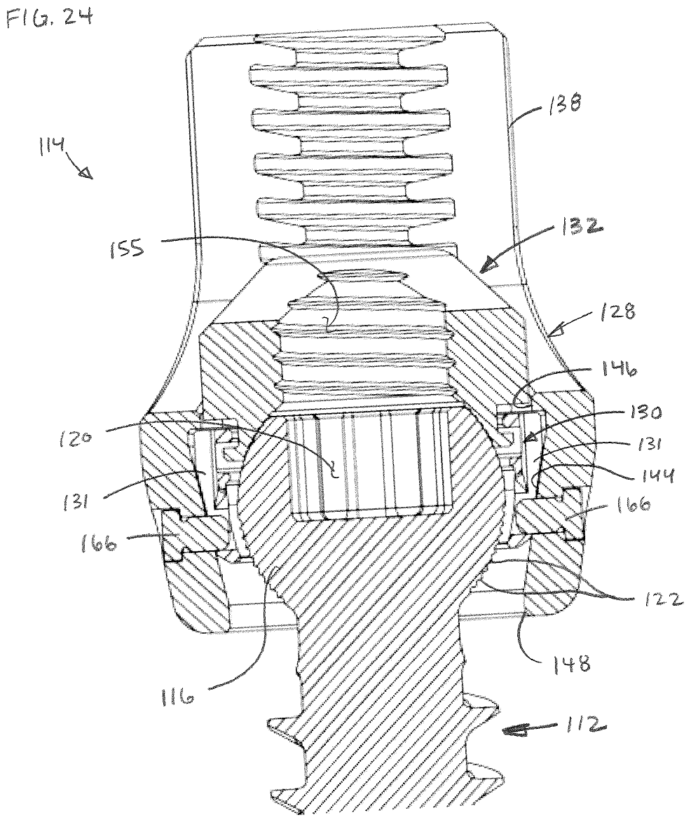

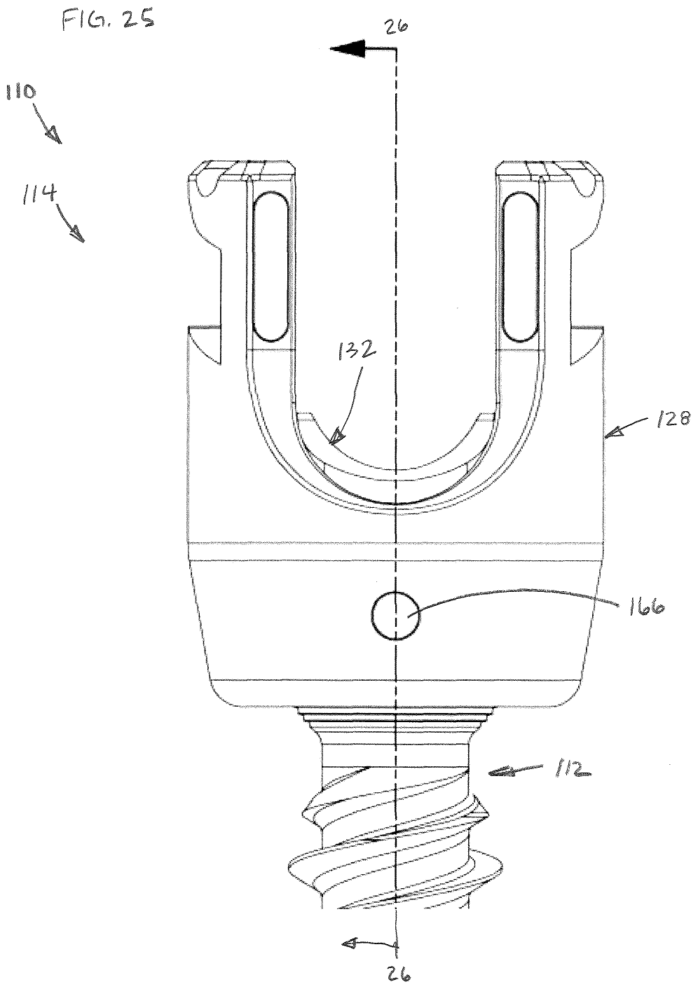

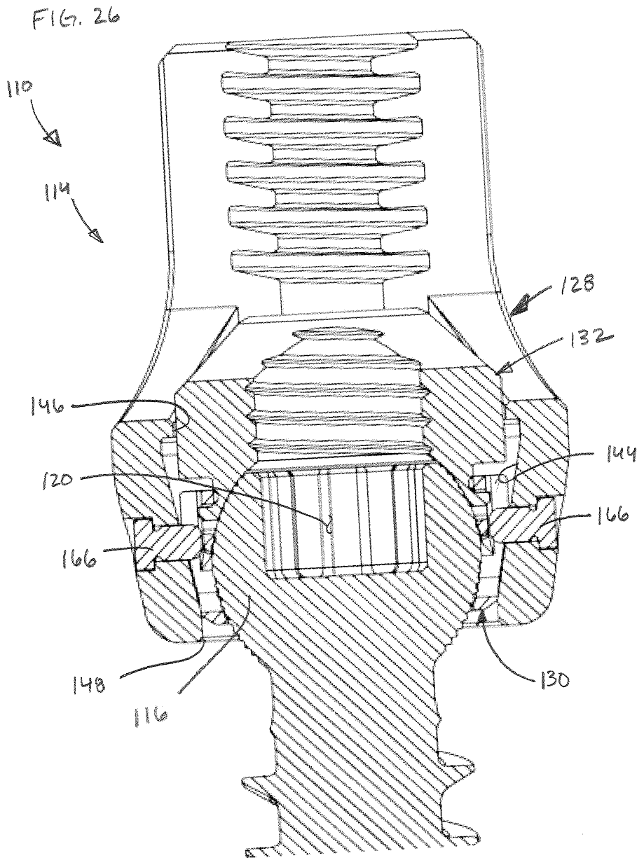

Referring to FIGS. 21 and 22, to secure the head assembly 114 to the fastener 112, the head 116 of the fastener is inserted into distal opening of the rod coupler 128. As the fastener head 116 is moved axially into the distal opening, the collet 130 moves proximally relative to the saddle 132, as explained above, and the collet 130 and saddle 132 also move proximally, generally together. Referring to FIGS. 23 and 24, as the fastener head 116 and the collet 130 and saddle 132 continue to advance axially (proximally), the collet 130 moves into the expansion cavity 144 of the rod coupler 128, whereupon the fastener head 116 pushes the ring portions of the collet radially apart and the fastener head enters (snaps into) the collet through the open distal end of the collet Referring to FIGS. 25 and 26, with the fastener head 16 received in the collet 130 in the head cavity, the saddle 132 and the collet 130 are moved distally (such as by using the tool secured to the saddle), whereupon the tapered distal interior surfaces of the rod coupler 128 push the ring portions toward one another to pinch the collet 130 against the fastener head 116 to inhibit the faster 112 from backing out of the head assembly 114. In this position, the head assembly 114 is capable of polyaxial movement relative to the fastener head 116 against the frictional forces of the friction rib 170 acting on the fastener head 116. The head assembly 114 is also capable of being removed from the fastener 112 using the tool by moving the saddle 132 and the collet 130 proximally relative to the rod coupler 128 such that the ring portions enter the expansion cavity. With the ring portions in the expansion cavity 144, the head assembly 114 can be moved proximally relative to the fastener head 116, causing the ring portions to move away from one another radially so that the fastener head is removed from the collet and the head assembly unsnaps or releases from the fastener head.

When the rod is secured to the screw 110, such as by threading the cap on the rod coupler 128, the head assembly 114 is fixed to the fastener 112 and inhibited from polyaxial movement when the rod is secured to the screw 10. Securing the rod to the screw 110 pushes the saddle 132 against the fastener head 116 and further compresses the collet 130 around the fastener head to inhibit movement of the head assembly 114 relative to the fastener head. The head assembly 114 may be secured to the fastener 112 before anchoring the fastener in bone or after anchoring the fastener in bone.

Modifications and variations of the disclosed embodiments are possible without departing from the scope of the invention defined in the appended claims.

When introducing elements of the present invention or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A spinal pedicle screw having proximal and distal ends and an axis extending therebetween, the spinal pedicle screw comprising:
a fastener comprising a shaft and a fastener head disposed on the shaft, the fastener being configured to be anchored within a subject's bone; and
a head assembly coupled to the fastener head, the head assembly including a rod coupler configured to secure a rod to the head assembly, and a collet received at least partially within the rod coupler,
wherein the fastener head is received in the collet,
wherein the collet has an interior surface engaging the fastener head,
wherein the collet includes a friction rib on the interior surface and protruding inward therefrom, the friction rib configured to impart a frictional force on the fastener head to inhibit free movement of the head assembly on the fastener head.

2. The spinal pedicle screw set forth in claim 1, wherein a distal portion of the interior surface of the collet engages the fastener head, the friction rib being disposed proximal of the distal portion of the interior surface of the collet.

3. The spinal pedicle screw set forth in claim 2, wherein the distal portion of the interior surface of the collet is concave.

4. The spinal pedicle screw set forth in claim 3, wherein the collet has proximal and distal open ends, wherein the friction rib is disposed between the proximal and distal open ends.

5. The spinal pedicle screw set forth in claim 4, wherein the head assembly further includes at least one pin received in a pin opening defined by the rod coupler, wherein the collet defines at least one opening in which the at least one pin is received to inhibit rotation of the collet about the axis relative to the rod coupler.

6. The spinal pedicle screw set forth in claim 5, wherein the head assembly further includes a saddle received in the rod coupler and connected to the collet, wherein the collet defines a concave interior surface engaging the fastener head.

7. The spinal pedicle screw set forth in claim 6, wherein the collet is axially movable relative to the saddle.

8. The spinal pedicle screw set forth in claim 7, wherein the collet is radially expandable within the rod coupler to enable insertion of fastener head into the collet, and removal of the fastener head from the collet.

9. The spinal pedicle screw set forth in claim 8, wherein the collet includes ring portions that are free from direct securement to one another and are in generally opposing relationship to effectively form a split ring.

10. The spinal pedicle screw set forth in claim 9, wherein the saddle defines at least one exterior groove, wherein each of the ring portions includes at least one tongue received in the at least one exterior groove, wherein the at least one tongue is movable axially within the at least one exterior groove.

11. A spinal pedicle screw having opposite proximal and distal ends and an axis extending therebetween, the spinal pedicle screw comprising:
a fastener comprising a shaft and a fastener head disposed on the shaft, the fastener being configured to be anchored within a subject's bone; and
a head assembly including
a rod coupler configured to secure a rod to the head assembly,
a collet received at least partially within the rod coupler, wherein the collet has an interior surface engaging the fastener head and at least one tongue extending radially inward from the interior surface, and
a saddle received at least partially within the rod coupler, the saddle having a proximal surface for seating the rod thereon, wherein the saddle defines at least one exterior groove,
wherein the at least one tongue of the collet is received the at least one groove of the saddle to couple the collet to the saddle,
wherein the collet and the saddle are movable axially relative to one another along the axis of the spinal pedicle screw.

12. The spinal pedicle screw set forth in claim 11, wherein a distal portion of the saddle is received in a proximal portion of the collet.

13. The spinal pedicle screw set forth in claim 11, wherein the collet is radially expandable within the rod coupler to enable insertion of fastener head into the collet through an open distal end of the collet, and removal of the fastener head from the collet through the open distal end of the collet.

14. The spinal pedicle screw set forth in claim 11, wherein the collet includes ring portions that are free from direct securement to one another and are in generally opposing relationship to effectively form a split ring.

15. The spinal pedicle screw set forth in claim 11, wherein the collet includes a friction rib on the interior surface and protruding inward therefrom, the friction rib configured to impart a frictional force on the fastener head to inhibit free movement of the head assembly on the fastener head.

16. The spinal pedicle screw set forth in claim 11, wherein the head assembly further includes at least one pin received in a pin opening defined by the rod coupler, wherein the collet defines at least one opening in which the at least one pin is received to inhibit rotation of the collet about the axis relative to the rod coupler.

17. A method of assembling the spinal pedicle screw set forth in claim 11, the method including:
inserting the collet into the rod coupler, wherein the collet is positioned in a first angular position about the axis of the spinal pedicle screw;
after said inserting the collet, inserting the saddle into the rod coupler such that a distal portion of the saddle is received in a proximal portion of the collet; and
rotating the collet 90 degrees from its first angular position to a second angular position so that the at least one tongue enters the at least one groove.

18. The method set forth in claim 17, further comprising:
inserting at least one pin into aligned openings defined by the rod coupler and the collet to inhibit rotation of the collet relative to the rod coupler.

19. A head assembly for a spinal pedicle screw having opposite proximal and distal ends and an axis extending therebetween, the head assembly comprising:
a rod coupler configured to secure a rod to the head assembly;
a collet received at least partially within the rod coupler, wherein the collet has an interior surface configured to engage the fastener head; and
a saddle received at least partially within the rod coupler, the saddle having a proximal surface for seating the rod thereon,
wherein together the collet and the saddle define a cavity sized and shaped to receive a fastener head of the fastener,
wherein the collet and the saddle are movable axially relative to one another along the axis of the spinal pedicle screw,
wherein the collet includes ring portions that are free from direct securement to one another and are in generally opposing relationship to effectively form a split ring,
wherein the ring portions are simultaneously movable axially and radially relative to the rod coupler to enable the fastener head of the fastener to be inserted into the collet as the head assembly is pushed down on the fastener head.

20. The head assembly set forth in claim 19, wherein the collet includes at least one tongue extending radially inward from the interior surface, wherein the saddle defines at least one exterior groove, wherein the at least one tongue of the collet is received in the at least one groove of the saddle to couple the collet to the saddle, wherein the collet and the saddle are movable axially relative to one another along the axis of the head assembly.
